(19) United States
(12) Patent Application Publication
(10) Pub. No.: US 2019/0388732 A1
(43) Pub. Date: Dec. 26, 2019

(54) Virtual Environment for Physical Therapy

(71) Applicant: International Business Machines Corporation, Armonk, NY (US)

(72) Inventors: Mauro Carlos PICHILIANI, Sao Paulo (BR); Marcia ITO, Sao Paulo (BR)

(21) Appl. No.: 16/014,540

(22) Filed: Jun. 21, 2018

(51) Int. Cl. A63B 24/00; G06K 9/00; G16H 20/30; G16H 10/60

(52) U.S. Cl. CPC A63B 24/0075; G06K 9/00342; A61B 5/0402; G16H 10/60; A63B 24/0062; G16H 20/30

(57) ABSTRACT

Method, system, and computer program product for a virtualized physical therapy system. The method may include initializing a virtualized physical therapy system for a patient and selecting actions to be performed by the patient during a physical therapy session. The method may also include detecting that the patient is performing the selected actions and displaying, a virtual representation of the selected actions. The method may additionally include monitoring the patient, using the virtualized physical therapy system, during the performance of the selected actions, updating one or more patient models with monitoring data, and generating a session evaluation report for the physical therapy session based on the patient models and the monitoring data.

### BACKGROUND

Physical therapy is often employed in the treatment of patients suffering from a variety of medical conditions where the patient has limited movement or pain in the movement of the patient's body. Technology, including sensors and virtual reality environments are often used to monitor a patient's movements and to aid a patient to view a movement in virtual reality. The present disclosure relates to physical therapy utilizing a virtual environment, and more specifically, to augmenting a physical therapy session with a virtual reality component, monitoring sensors, and a brain computer interface to provide an enhanced virtual reality physical therapy session.

### SUMMARY

According to one embodiment of the present invention a method is provided. The method may include initializing a virtualized physical therapy system for a patient. The virtualized physical therapy system may include a brain computer interface (BCI) connected to the patient, a virtual reality component, and one or more monitoring sensors. The method may further include selecting one or more actions to be performed by the patient using the virtualized physical therapy system during a physical therapy session, detecting, using the BCI, that the patient is performing one or more of the selected actions, and displaying, using the virtual reality component, a virtual representation of the selected actions. The method may also further include monitoring the patient, using the virtualized physical therapy system, during the performance of the selected actions, updating one or more patient models with monitoring data, and generating a session evaluation report for the physical therapy session based on the patient models and the monitoring data.

According to one embodiment a virtualized physical therapy system is provided. The system may include processing resources, a brain computer interface (BCI) connected to a patient, a virtual reality component, one or more monitoring sensors, one or more computer processors, and a memory containing a program which when executed by the one or more computer processors performs an operation. The operation may include initializing the virtualized physical therapy system for a patient. The operation may further include selecting one or more actions to be performed by the patient using the virtualized physical therapy system during a physical therapy session, detecting, using the BCI, that the patient is performing one or more of the selected actions, and displaying, using the virtual reality component, a virtual representation of the selected actions. The operation may also further include monitoring the patient, using the virtualized physical therapy system, during the performance of the selected actions, updating one or more patient models with monitoring data, and generating a session evaluation report for the physical therapy session based on the patient models and the monitoring data.

According to one embodiment a computer program product is provided. The computer program product may include a computer-readable storage medium having computer-readable program code embodied therewith, the computer-readable program code executable by one or more computer processors to perform an operation. The operation may include initializing a virtualized physical therapy system for a patient. The virtualized physical therapy system may include a brain computer interface (BCI) connected to the patient, a virtual reality component, and one or more monitoring sensors. The operation may further include selecting one or more actions to be performed by the patient using the virtualized physical therapy system during a physical therapy session, detecting, using the BCI, that the patient is performing one or more of the selected actions, and displaying, using the virtual reality component, a virtual representation of the selected actions. The operation may also further include monitoring the patient, using the virtualized physical therapy system, during the performance of the selected actions, updating one or more patient models with monitoring data, and generating a session evaluation report for the physical therapy session based on the patient models and the monitoring data.

### BRIEF DESCRIPTION OF THE SEVERAL VIEWS OF THE DRAWINGS

FIG. 1 is an example physical therapy virtual environment system, according to one embodiment.

FIG. 2 illustrates an example process for a physical therapy session using a physical therapy virtual environment system, according to one embodiment.

FIG. 3 is a block diagram of a server, such as computer 102, to facilitate a virtualized physical therapy system, according to one embodiment.

FIG. 4 illustrates a method for a virtualized physical therapy system, according to one embodiment.

FIG. 5 illustrates a method for calibrating components of a virtualized physical therapy system, according to one embodiment.

FIG. 6 illustrates a method for selecting actions to perform in a virtualized physical therapy system, according to one embodiment.

FIG. 7 illustrates a method for detecting that a patient is performing selected actions, according to one embodiment.

FIG. 8 illustrates a method for displaying virtual representations of selected actions, according to one embodiment.

FIG. 9 illustrates a method for monitoring a patient, according to one embodiment.

FIG. 10 illustrates a method for providing feedback to a patient, according to one embodiment.

FIG. 11 illustrates a method for evaluating a physical therapy session, according to one embodiment.

FIG. 12 illustrates an additional method for a virtualized physical therapy system, according to one embodiment.

### DETAILED DESCRIPTION

Physical therapy (PT) is a process used by many health care providers to help patients rehabilitate or strengthen parts of the patient's body that have been injured or weakened. For example, acute motor deficit is a debilitating condition that affects a significant percentage of patients that suffer from neurological disorders, amputations, arthritis, stroke, and other related medical conditions. In some instances, patients with motor deficit also experience chronic pain and unpleasant sensations, such as ache, burn or skin puncture. PT (among other therapeutic approaches) is used to alleviate the symptoms and prepare the patients' mental state to either receive a prosthetic limp or regain the natural motor ability. In some cases, a health care provider performs a treatment protocol including multiple PT sessions to reach the desired motor rehabilitation goals. The PT sessions may include resources such as mirror boxes, plastic balls, sensors, and other specialized equipment that are combined with the expertise, experience and knowledge of the health care provider to ameliorate, reduce, treat or eliminate symptoms. Enhancing a patient's treatment with a brain computer interface, virtual reality components, and monitoring sensors will aid the health care provider by providing vital information in treating the patient and adjusting/developing the treatment protocol for the patient.

As described herein, methods and systems to apply a combination of virtual reality environments, brain computer interfaces with cognitive guidance, and monitoring sensors aid in physical therapy treatment. For example, the virtual reality components described herein provide visual feedback as a virtual body part that moves to reflect the motion effort performed by the patient. Further, the brain computer interface described herein configured receive and interpret brain signals from the patient and correlate the signals controlling and moving a virtual body part, in a virtual reality component. Cognitive guidance from the brain computer interface and the monitoring sensors attached to the patient may also be used to help the preparation and execution of the PT session, including exercise sequence information, proper movement angles and position, time limitation, amount of repetitions, metrics and thresholds to exercise's interruption, and general/specific evaluation of the treatment to maximize the rehabilitation goals defined by the health care provider and the patient. The system may also be configured to learn from each PT session and adapt the treatment protocols and session factors to accommodate the factors that affected the overall performance of the past treatments when the system was used in treatment.

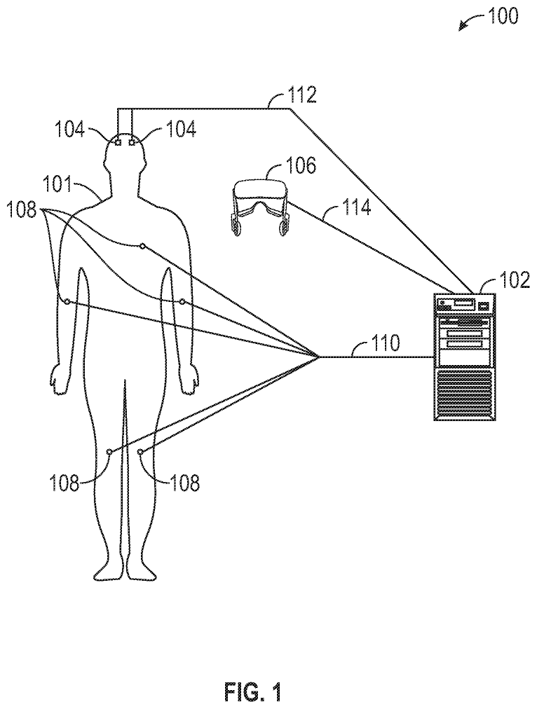

With reference now to FIG. 1, which depicts a physical therapy virtual environment system 100, according to one embodiment. As shown, the system 100 is generally configured to aid or augment a physical therapy session for a patient 101. The system 100 includes a computer 102. The computer 102 may be embodied in the form of a general-purpose computing device. The computer 102 may also be embodied as a mobile device, such as a smart phone or tablet, or a device specially configured for the physical therapy virtual environment system. As shown, the computer 102 is connected to a brain computer interface system 104 (BCI 104). Generally, the BCI is configured to detect and interpret brain waves or signals of patient 101. In the depicted embodiment, the BCI transmits detected and/or interpreted brain signal data to the computer 102 via a connection 112.

System 100 also includes a virtual reality component 106. In some examples, the virtual reality component 106 comprises a virtual reality headset that is configured to fit on the head of patient 101 and provide a virtual reality experience to the patient 101. The virtual reality component 106 also comprises one of another virtual reality devices configured to provide a virtual reality experience to the patient 101. As shown, the virtual reality component 106 is connected to the computer 102 via connection 114.

System 100 also includes one or more sensors 108 connected to the computer 102 via a connection 110. Sensors 108 comprise biometric sensors configured to provider information about the physical and mental state of patient 101. In some examples, the sensors 108 include heart rate monitors, such as an Electrocardiography (ECG) sensors, oximeters, motion sensors, force sensors, thermometers, and other body measurement devices and sensors.

As shown in FIG. 1, connections 110, 112, and 114 comprise a wired or wireless connection. For example, the various connections may be directly wired into computer 102 via unique device specific connections or may be connected via universal serial bus (USB), Ethernet, fiber optic connections, or other connection industry standards. The connections may also comprise wireless connections to computer 102 via wireless standards such as Wi-Fi, Bluetooth, Bluetooth LE, etc.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

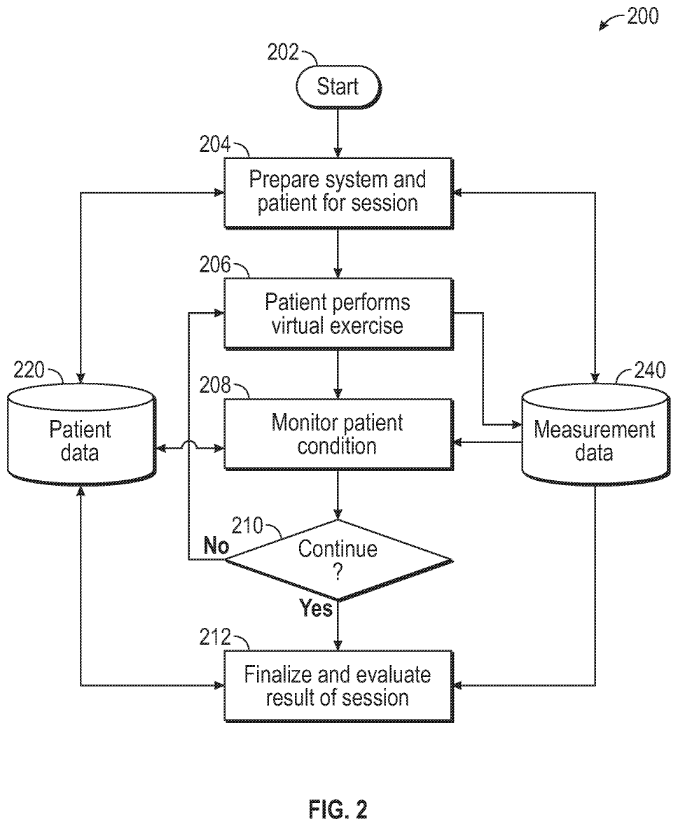

FIG. 2 illustrates an example process 200 for a physical therapy session using a physical therapy virtual environment system, according to one embodiment. The process 200 utilizes a physical therapy virtual environment system such as system 100 described in relations to FIG. 1. As shown, the process starts at block 202 and proceed to block 204 where the system and patient are prepared for the physical therapy session. In some examples, a health care provider, such as a physical therapist, manipulates both the system 100 and the patient 101 in order to ensure that the system is configured correctly and that the patient is connected to the system in order to ensure correct functioning of the system 100. While examples described herein generally refer to a healthcare provider and a patient both using the system 100, in some examples, a patient 101 operates system 100 independently. In some examples, the healthcare provider is located remotely from the patient 101 and interacts with the patient through system 100, such as through the virtual reality component 106.

Block 204 also includes, having the patient physically prepare for the physical therapy session. For example, the patient needs to be prepared by warming up or other body activities such as stretching or dynamic movement before the physical therapy session begins. This warming up period may be directed by the healthcare provider. In some examples, the system 100 provides instructions to the healthcare provider and/or the patient 101 for warming up.

In some examples, preparing the system also includes initializing the virtualized physical therapy system 100 for the patient 101. For example, the system 100 is configured to determine a proper positioning for the brain controlled interface, the virtual reality component, and the one or more monitoring sensors, indicate the proper positioning to the healthcare provider/patient, and perform one or more calibration methods to calibrate the virtualized physical therapy system to the patient. In some examples, the system 100 determines a proper positioning for the brain controlled interface, the virtual reality component, and the one or more monitoring sensors. This may also include setting and calibrating BCI 104 according to specifications which are set by the BCI 104 and/or by computer 102. Likewise, virtual reality component 106 may also be set and calibrated to the patient 101 according to specifications which set by the virtual reality component 106 and/or by computer 102. The monitoring sensors 108 may also be placed and calibrated according to specifications based on the type of sensors and set by computer 102 for the system 100.

In some examples, the system 100 needs to be initialized and configured to patient 101 during the first use of the system 100 with patient 101. In some examples, subsequent uses of the system 100 utilize patient 101's stored configuration information, stored in computer 102.

As also shown in FIG. 2 the process then proceeds to block 206, where the patient performs virtual exercises. In some examples, the system 100 first selects one or more actions to be performed by the patient using the virtualized physical therapy system during the physical therapy session. In some examples, the healthcare provider/patient selects one or more actions to perform the exercises. In some examples, the system 100 selects the one or more actions from a stored physical therapy plan stored on the system 100 and/or based on past physical therapy sessions. For example, the system may receive action input from a user (e.g., the patient and/or healthcare provider) and determine from the action input, the one or more actions to perform.

In another example, the system 100 chooses a virtual exercise based on the recommendation of the last session (e.g., a system recommendation in a stored plan and/or a recommendation made by the healthcare provider). In some examples, the system 100 is configured to first explain to the patient how to interact in and with the virtual environment such as how to perform simple or test actions and exercises. Once the patient is comfortable with the virtual environment the system then explains how to perform the virtual exercise using audio and the virtual display. For example, the system 100 determines a virtual representation of the selected action/exercise and display the virtual representation on a patient display of the virtual reality component 106, which comprises a virtual reality headset.

In some examples, the system 100 also displays using the virtual reality component 106 the effect or movement of the action/exercise as the patient performs the movement. In some examples, the system 100 determines and provides feedback to the patient during the performance of the actions/exercise (e.g., adjust the action, repeat the action, demonstrate alternate performances, positive reinforcement, etc.). The system 100 displays the feedback and performance of the action to the healthcare provider during the exercise and also displays the result of the action/exercise after completion by the patient.

The process then proceeds to block 208, where the system 100 is configured to monitor the patient's physiological and psychological conditions in order to prevent injury to the patient. In some examples, the system 100 monitors the patient, using the virtualized physical therapy system (e.g., system 100), during the performance of the selected actions/exercises and update one or more patient models with monitoring data. The patient models comprise patient data 220 and measurement data 240 shown in FIG. 2. Patient data 220 includes Clinical data (patient structured data from electronic medical records (EMR), electronic health records (EHR), hospital information systems (HIS) data and patient generated health data (PGHD) (health-related data created, recorded or gathered by or from patients, family member, or other caregivers) to help address a health concern. PGHD includes: (1) health history, (2) treatment history; (3) biometric data; (4) symptoms and (5) lifestyle choices. PGHD stored in patient models may be distinguished from data generated in clinical settings and through encounters with a healthcare provider since patients, not providers, are primarily responsible for capturing or recording these data and patients decide how to share or distribute these data to health care providers and others. The patient data including the PGHD may be collected from sources outside of system 100 or may be inputted into system 100 by patient 101 (or another user) using input/output devices connected to system 100. Measurement data 240 includes the measurements of the actions/exercises from BCI 104, and sensors 108, etc.

In some examples, the system 100 and/or the healthcare provider may query the patient 101 on the effects of the actions/exercises including pain, discomfort, etc. In some examples, the sensors 108 comprise one or more monitoring sensors including biometric sensors configured to read biometric indications from the patient. The system 100 monitor the patient, by receiving, from the biometric sensors (sensors 108) and BCI 104, biometric data representing a current physical condition of the patient; receiving, from the biometric sensors and BCI, biometric data representing a current physiological condition of the patient, and generating a current monitoring score (which may include and/or indicate a patient discomfort level) for the patient. For example, if the sensors 108 and BCI 104 detect increased blood pressure and distress signals in the brain of the patient 101, the system 100 generates a high monitoring score indicating that the patient is experiencing discomfort or pain. If the sensors 108 and BCI 104 indicate that the patient does not show signs of increased discomfort (e.g., normal blood pressure and brain signals), the system 100 generates a lower monitoring score.

The process then proceeds to block 210, where the system 100 is configured to determine if the selected actions and/or the physical therapy session should continue. For example, during the performance of the selected actions, the system 100 selects a stop session threshold such as a time limit or a discomfort level. If the system 100 determines that the stop session threshold has not been met, the system may instruct the patient 101 and healthcare provider to continue the physical therapy session. For example, if the monitoring score indicates that patient 101 does not have too much discomfort or if timer started at the beginning of the physical therapy session has not expired, the system 100 will continue the physical therapy session. In some examples, upon determining that the continuing session threshold has been met, the system 100 stops the physical therapy session. For example, if the timer has expired and/or the monitoring score indicates the patient 101's discomfort is too high, the system 100 will end the physical therapy session.

In some examples at block 212, the system 100 includes a wind down or cool down phase once the physical therapy session has ended in order to acclimate the patient 101 back to the non-virtual world. For example, system 100 may slowly reveal the non-virtual world back to the patient 101. In some examples, the system 100 then generates a session evaluation report for the physical therapy session based on the patient models (patient data 220) and the monitoring data (measurement data 240). The system 100 also generates a report including one or more actions to be performed for a future physical therapy session determined from the patient data 220 and measurement data 240.

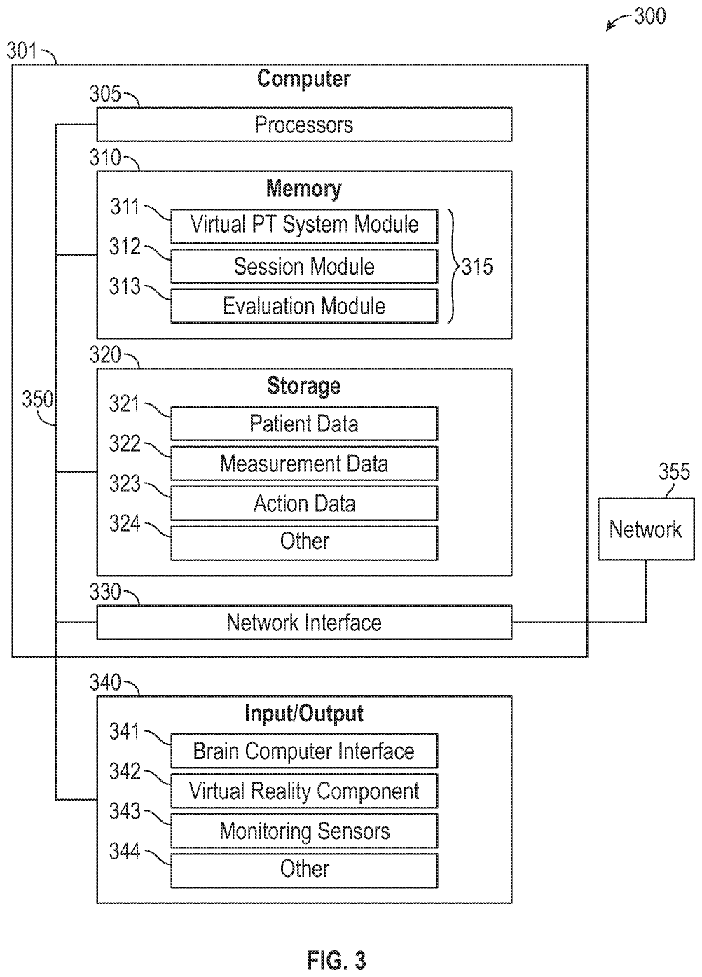

FIG. 3 is a block diagram of a server, such as computer 102, to facilitate a virtualized physical therapy system, according to one embodiment. As shown in FIG. 3, the arrangement 300 includes the computer 102 embodied as computer 301 configured to execute the function of the system 100 including executing and/or augmenting the process 200. The computer 301 is shown in the form of a general-purpose computing device. The components of computer 301 include, but are not limited to, one or more processors or processing units 305, a system memory 310, a storage system 320, network interface 330, and a bus 350 that couples various system components including the system memory 310 and storage system 320 to processors 305 along with various input/output (I/O) components 340. In other embodiments, arrangement 300 is distributed and includes a plurality of discrete computing devices that are connected through wired or wireless networking.

In some examples, I/O components 340 include a brain computer interface 341, a virtual reality component 342, monitoring sensors 343, and other input/output components 344. The other components 344 may comprise a keyboard, mouse, touch screen display, or other input/output components configured to allow a user (e.g., a patient and or healthcare provider) to input information to the computer 301. Computer 301 also communicates with one or more external devices such as a keyboard, a pointing device, a display, etc.; and one or more devices that enable a user to such as interact with computer 301; and/or any devices (e.g., network card, modem, etc.) that enable computer 301 to communicate with one or more other computing devices locally and/or remotely via network interface 330 and network 355. Such communication can also occur via I/O components 340. Additionally, computer 301 may communicate with one or more networks such as a local area network (LAN), a general wide area network (WAN), and/or a public network (e.g., the Internet) via network interface 330. As depicted, network interface 330 communicates with the other components of computer 301 via bus 350. It should be understood that although not shown, other hardware and/or software components could be used in conjunction with computer 301. Examples, include, but are not limited to: cloud computing systems, microcode, device drivers, redundant processing units, external disk drive arrays, RAID systems, tape drives, and data archival storage systems, etc.

Bus 350 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer 301 typically includes a variety of computer system readable media. Such media may be any available media that is accessible by computer 301, and it includes both volatile and non-volatile media, removable and non-removable media. System memory 310 can include computer system readable media in the form of volatile memory, such as random access memory (RAM) and/or cache memory. Computer 301 may further include other removable/non-removable, volatile/non-volatile computer system storage media. By way of example, storage system 320 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 350 by one or more data media interfaces. As will be further depicted and described below, memory 310 may include at least one program product having a set (e.g., at least one) of program modules 315 that are configured to carry out the functions of embodiments of the disclosure described herein.

Storage 320 also includes media for storing patient data 321, including the patient models and patient data 220. The storage system 320 also includes measurement data 322, which can include measurement data 240, action data 323 which includes exercises and other action data for selection, including visual representations of the actions, and other data 324 related to the system 100. The various data 321-324 is updated and accessed by the program modules 315 described herein, including the virtual physical therapy (PT) module 311, the session module 312, and the evaluation module 313. Storage system 320 also includes other information stored for access and use by the computer 301.

Memory 310 includes a plurality of program modules 315 for performing various functions described herein. The modules 315 generally include program code that is executable by one or more of the processors 305. As shown, modules 315 include virtual PT module 311, session module 312, and evaluation module 313.

The program modules 315 also interact with each other and storage system 320, and the I/O components 340 to perform certain functions. For example, virtual PT system module 311 may be configured to initialize a virtualized physical therapy system for a patient. In some examples, virtual PT system module 311 initiates system 100 including a brain computer interface (e.g., BCI 341 embodied as BCI 104) connected to the patient 101, virtual reality component (e.g., virtual reality component 342 embodied as virtual reality component 106), and one or more monitoring sensors (e.g., monitoring sensors 343 embodied as sensors 108).

In one example, the virtual PT system module 311 uses the I/O components 340 and other data 324 to determine a proper positioning for the brain controlled interface 341, the virtual reality component 342, and the one or more monitoring sensors 343 on the patient 101. Virtual PT system module 311 also indicates the proper positioning to a user (e.g., patient 101 and/or a healthcare provider) and performs one or more calibration methods to calibrate the virtualized physical therapy system to the patient. These calibration methods may be provided by the manufacturer of the various I/O components 340 and stored as other data 324.

Additionally, virtual PT system module 311 detects, using the BCI, that the patient is performing one or more selected actions. For example, virtual PT system module 311 uses BCI 341 embodied as BCI 104 to detect that the patient 101 is performing the actions. In some examples, virtual PT system module 311 is configured to receive an indication from the BCI 341 embodied as BCI 104 that the patient 101 is performing a movement and determine, using action data 323, that the movement corresponds to the selected action. In some examples, the BCI 104 sends data to the computer 102 which then determines if the measured mental effort of the patient 101 is enough or not to perform the selected action. In some examples, the virtual PT system module 311 may then compare the mental effort performed for the selected action with stored BCI data, such as data gathered during a calibration of the BCI 104. If the mental effort is above a threshold set by the calibrated data the movement is made and the patient 101 will see the virtual completion of the selected action using virtual reality component 106 (e.g., a virtual leg moving). Otherwise, the virtual performance of selected action will not appear to the user using the virtual reality component 106, indicating more effort is needed.

The virtual PT system module 311 is configured to display, using the virtual reality component, a virtual representation of the selected actions. For example, virtual PT system module 311 utilizes virtual reality component 342 embodied as virtual reality component 106 to display the virtual representation of the selection actions. In some examples, the virtual PT system module 311 also determines a virtual representation of the selected action from action data 323 and displaying the virtual representation on a patient display of the virtual reality component 342 embodied as virtual reality component 106. In some examples, virtual reality component 342 embodied as virtual reality component 106 may comprise a virtual reality headset configured to fit over the head and/or eyes of patient 101. In some examples, the virtual representation depends on the patient. For example, if a patient 101 is missing a limb or cannot physically move the limb, the virtual representation comprises a virtual presentation of the missing limb. For example, the virtual representation may comprise a leg kicking a ball, when the selected action is to kick a ball (and the BCI 104 has detected the patient is performing the action). In some examples, the virtual representations correlate to the specific patient body part, i.e. a male leg for male patients or female arm for female patients, etc.

Additionally, the virtual PT system module 311 monitors the patient, using the virtualized physical therapy system, during the performance of the selected actions. For example, the virtual PT system module 311 monitors, using the BCI 341 embodied as BCI 104, the monitoring sensors 343 embodied as sensors 108, and virtual reality component 342 embodied as virtual reality component 106, the physical and psychological state of patient 101 performance of the selected actions as they are being performed both virtually and physically by patient 101.

For example, the monitoring sensors 343 embodied as sensors 108 comprise biometric sensor and the virtual PT system module 311 receives, from the biometric sensors (e.g., monitoring sensors 343 embodied as sensors 108) and BCI 341 embodied as BCI 104, biometric data representing a current physical condition of the patient and receive, from the biometric sensors (e.g., monitoring sensors 343 embodied as sensors 108) and BCI 341 embodied as BCI 104, biometric data representing a current psychological condition of the patient. The virtual PT system module 311 uses the received biometric data along with patient data 321, other data 324, and data input from a user (e.g., patient 101 and/or a healthcare provider) to determine the physical and psychological condition. For example, increased heart rate, blood pressure, and other indications in the received biometric data indicates increased levels of pains and/or stress in the patient 101. The virtual PT system module 311 then uses the received biometric data, the patient data 321, and other data 324 to generate a current monitoring score for the patient using the received biometric data and additional data. The virtual PT system module 311 then updates one or more patient models including patient data 321 and measurement data 322 with monitoring data including the received biometric data, user data, and the current monitoring score.

Furthermore, the virtual PT system module 311, during the performance of the selected actions, determines feedback on the performance of the selected actions and provides the feedback to the patient using the virtual reality component. For example, virtual PT system module 311 determines feedback from the patient data 321, measurement data 322, and action data 323 to provide to the patient. This may include improving the action form, increase action effort, or other relevant feedback. The virtual PT system module 311, utilizing virtual reality component 342 embodied as virtual reality component 106, provides the feedback to the patient 101. For example, the virtual reality component 106 indicates to patient 101 to correct form, increase effort, etc.

As described above the program modules 315 include session module 312. In one example, the session module 312 selects one or more actions to be performed by the patient using the virtualized physical therapy system during a physical therapy session. For example, the session module 312, using patient data 321, action data 323, and other data 324 selects one or more actions of the action data 323 for the patient 101 to perform during the physical therapy session. In some examples, the session module 312 receives action input from a user via I/O components 340. The user may include a healthcare provider or patient 101. The session module 312 then determines from the action input, the one or more actions to perform. For example, a user selects individual actions to perform, a group or list of actions to perform, one or more actions from a stored physical therapy plan, etc.

Additionally, the session module 312 during the performance of the selected actions, selects a stop session threshold. For example, the session module 312 selects a stop session threshold from other data 324. In some examples, the stop session threshold comprises a patient discomfort threshold or a session time threshold. The session module 312 also, upon determining that the stop session threshold has not been met, continues the physical therapy session. For example, the session module 312 allows the next action to be presented and performed by the patient 101. The session module also, upon determining that the continuing session threshold has been met, stops the physical therapy session. For example, the session module 312 enters into a cool down period or stop the physical therapy session.

In an example when the stop session threshold is a patient discomfort threshold, the session module 312 determines from the patient models (such as patient data 321 and the current monitoring score) that a current patient discomfort level is below the patient discomfort threshold (as stored in other data 324) and/or determine from the patient models that the current patient discomfort level is above the patient discomfort threshold. For example, if the patient is in a low or acceptable level of discomfort, the session will continue, but if the patient is in a high or unacceptable level of discomfort the session will terminate or stop. In another example, when the stop session threshold is a session time threshold, the session module 312 determines from a session timer started at the beginning of the physical therapy session that a current session time is below the session time threshold and/or determines from the session timer that the current session time is above the session time threshold. For example, as the physical therapy session begins, session module 312 begins a timer. After each action is performed the session module 312 verifies that the timer has not exceeded the threshold (e.g., 60 minutes). Once the threshold is met, the session module 312 begins terminating the physical therapy session.

Furthermore, the program modules 315 also includes evaluation module 313 configured to evaluate and finalize a physical therapy session. In some examples, the evaluation module 313 generates a session evaluation report for the physical therapy session based on the patient models and the monitoring data. For example, the evaluation module 313 using patient data 321 and measurement data 322 generates a session evaluation report including which exercises and actions were performed by patient 101, how the actions were performed by patient 101, and other information about the physical therapy session. In some examples, the evaluation module 313 generates a final score (such as a level of completeness, etc.) for the selected actions performed using the one or more patient models and measurement data and determine, from the one or more patient models, one or more actions to be performed for a future physical therapy session. For example, if an action was difficult for the patient 101 to complete, the action may be altered, included, and/or adjusted for the next session. If an action was not difficult, a more challenging action or increased repetitions of the action may be included in the actions to be performed in the future physical therapy session.

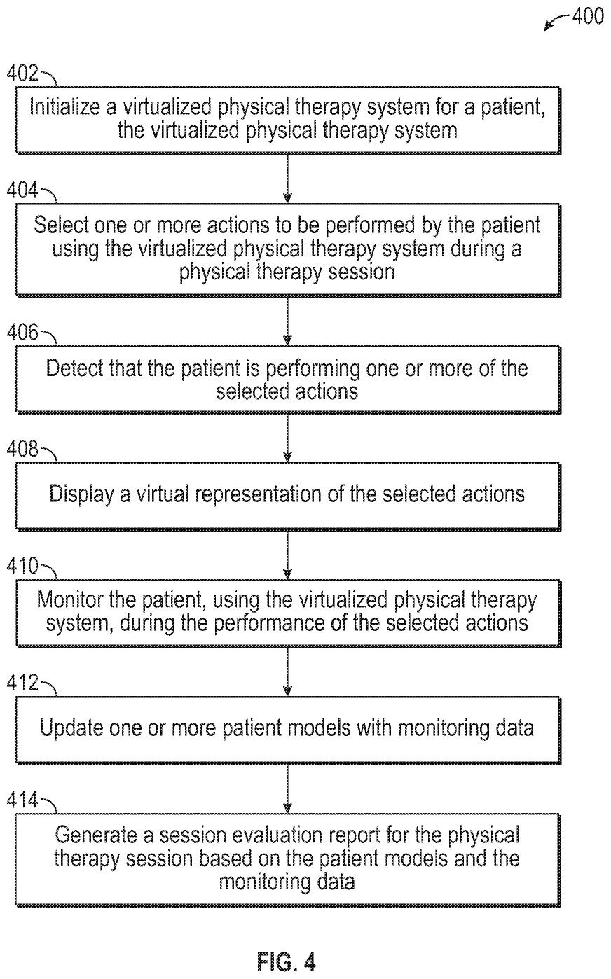

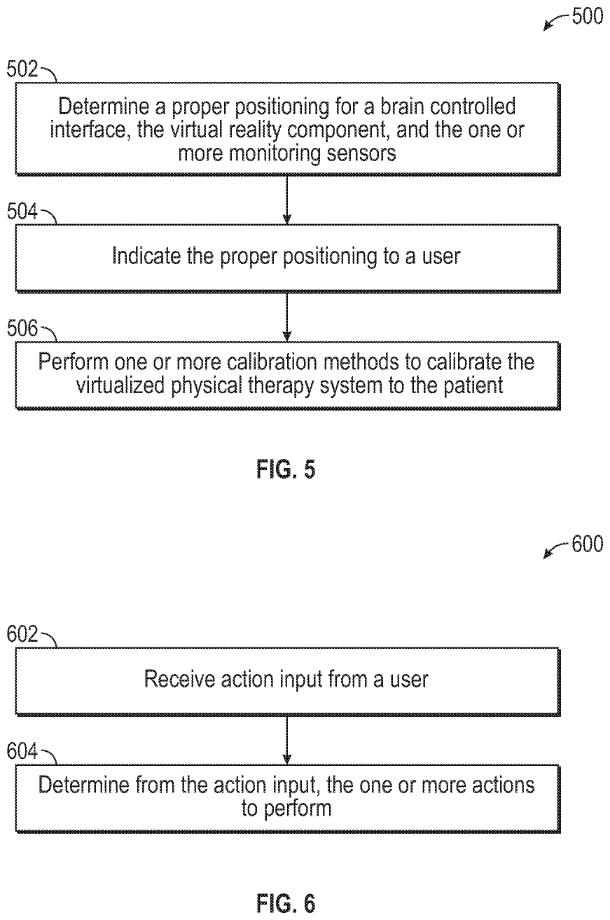

FIG. 4 illustrates a method for a virtualized physical therapy system, according to one embodiment. Method 400 begins at block 402 where a computer, such as computer 301, initializes a virtualized physical therapy system for a patient. In some examples, the virtualized physical therapy system includes a brain computer interface (BCI) connected to the patient, a virtual reality component, and one or more monitoring sensors as discussed in relation to FIGS. 1-3. Furthermore, an additional method for initializing the virtualized physical therapy system is described in relation to FIG. 5.

Method 400 continues at block 404, where a computer, such as computer 301, selects one or more actions to be performed by the patient using the virtualized physical therapy system during a physical therapy session. In some examples, selecting the one or more actions to be performed by the patient during a physical therapy session includes selecting the one or more actions from a stored physical therapy plan. An additional method for selecting one or more actions to be performed by the patient during a physical therapy session is described in relation to FIG. 6.

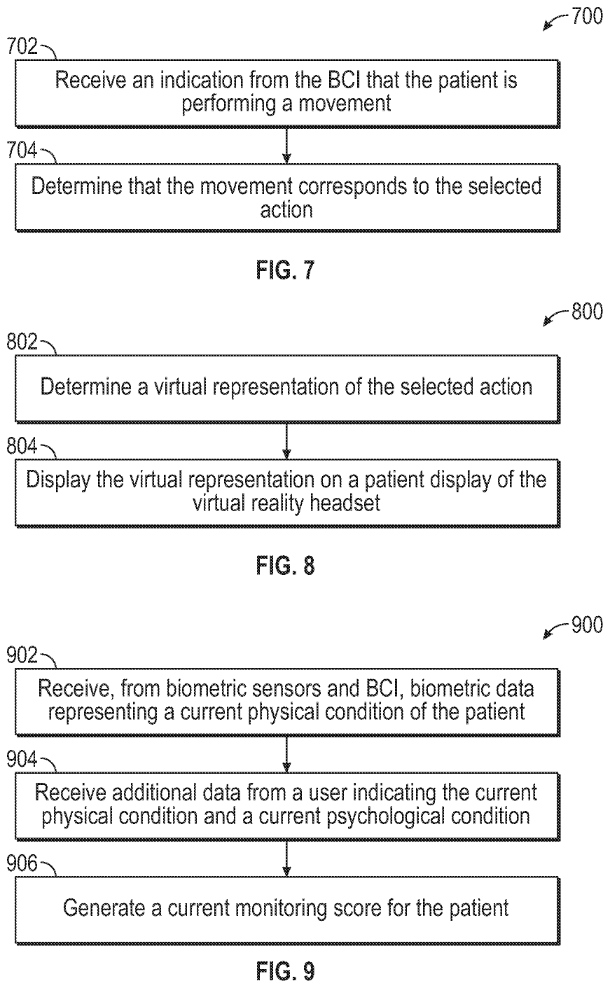

At block 406, method 400 continues where a computer, such as computer 301, detects, using the BCI, that the patient is performing one or more of the selected actions. An additional method for detecting, using the BCI, that the patient is performing one or more of the selected actions is described in relation to FIG. 7. The method 400 then continues at block 408 where a computer, such as computer 301, displays, using the virtual reality component, a virtual representation of the selected actions. An additional example method for displaying, using the virtual reality component, a virtual representation of the selected actions is described in relation to FIG. 8.

At block 410, method 400 continues where a computer, such as computer 301, monitors the patient, using the virtualized physical therapy system, during the performance of the selected actions. An additional example method for monitoring the patient, using the virtualized physical therapy system, during the performance of the selected actions is described in relation to FIG. 9. The method 400 then continues at block 412 where a computer, such as computer 301, updates one or more patient models with monitoring data and at block 414, where a computer such as computer 301 generates a session evaluation report for the physical therapy session based on the patient models and the monitoring data. An additional example method for generating a session evaluation report for the physical therapy session based on the patient models and the monitoring data described in relation to FIG. 11.

FIG. 5 illustrates a method for calibrating components of a virtualized physical therapy system, according to one embodiment. Method 500 begins at block 502 where a computer, such as computer 301, determines a proper positioning for the brain controlled interface, the virtual reality component, and the one or more monitoring sensors. In some examples, the BCI 104 and the virtual reality component are initially placed on the patient 101. The devices may then along with computer 102 may communicate positioning and calibration data. For example, the BCI 104 may determine a set of calibration steps for the user to perform, such as concentrating on a specific mental image for a period of time. The virtual reality component may also determine a more appropriate position based on user actions and feedback.

Method 500 continues at blocks 504 and 506 where a computer, such as computer 301, indicates the proper positioning to a user, and performs one or more calibration methods to calibrate the virtualized physical therapy system to the patient. For example, the BCI 104, virtual reality component 106, and the computer 102 may communicate the calibration means to the patient 101 and/or a healthcare provider both during calibration steps and as a final output on where to position the devices.

FIG. 6 illustrates a method for selecting actions to perform in a virtualized physical therapy system, according to one embodiment. Method 600 begins at block 602 where a computer, such as computer 301, receives action input from a user. Method 600 continues at blocks 604 where a computer, such as computer 301, determines from the action input, the one or more actions to perform. In some examples, the user may comprise a patient and/or a healthcare provider that is able to construct a physical therapy session, built from the actions to perform and based on the action input into the system.

FIG. 7 illustrates a method for detecting that a patient is performing selected actions, according to one embodiment. Method 700 begins at block 702 where a computer, such as computer 301, receives an indication from the BCI that the patient is performing a movement. Method 700 continues at block 704 where a computer, such as computer 301, determines that the movement corresponds to the selected action.

FIG. 8 illustrates a method for displaying virtual representations of selected actions, according to one embodiment. In some examples, the virtual reality component comprises a virtual reality headset. The method 800 begins at block 802 where a computer, such as computer 301, determines a virtual representation of the selected action. Method 800 continues at block 804 where a computer, such as computer 301, displays the virtual representation on a patient display of the virtual reality headset.

FIG. 9 illustrates a method for monitoring a patient, according to one embodiment. In some examples, monitoring sensors used to monitor the patient comprises biometric sensors. The method 900 begins at block 902 where a computer, such as computer 301, receives, from the biometric sensors and BCI, biometric data representing a current physical condition of the patient. Method 900 continues at block 904 where a computer, such as computer 301, receives, from the biometric sensors and BCI, data representing a current physical condition of the patient. Method 900 then continues at block 906 where a computer, such as computer 301, receives additional data from a user indicating the current physical condition and a current psychological condition. Method 900 then proceeds to block 906 where a computer, such as computer 301, generates a current monitoring score for the patient using the data and additional data.

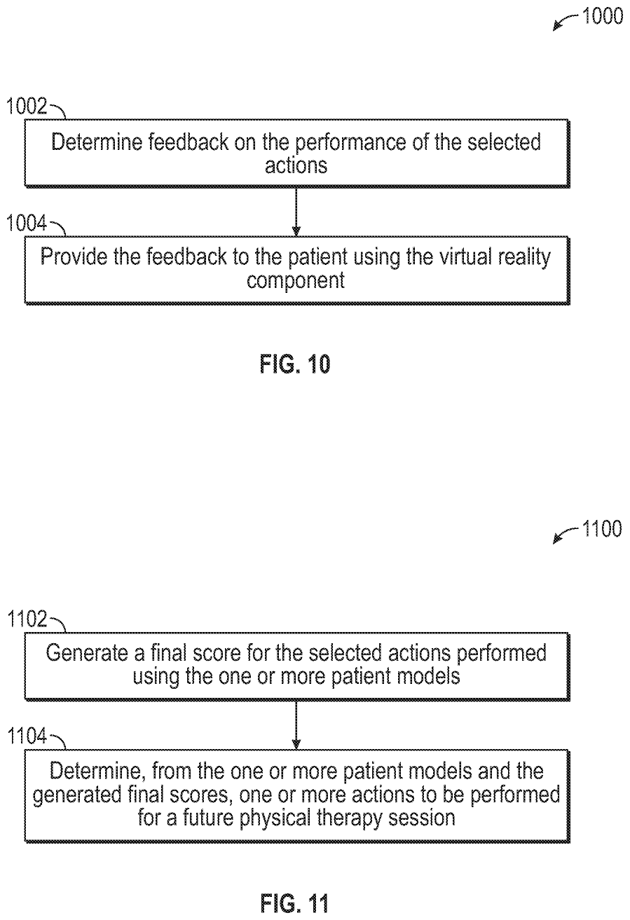

FIG. 10 illustrates a method for providing feedback to a patient, according to one embodiment. The method 1000 begins at block 1002 where a computer, such as computer 301, determines feedback on the performance of the selected actions during the performance of the selected actions by the patient. Method 1000 continues at block 1004 where a computer, such as computer 301, provides the feedback to the patient using the virtual reality component.

FIG. 11 illustrates a method for evaluating a physical therapy session, according to one embodiment. The method 1100 begins at block 1102 where a computer, such as computer 301, generates a final score for the selected actions performed using the one or more patient models. Method 1100 continues at block 1104 where a computer, such as computer 301, determines, from the one or more patient models, one or more actions to be performed for a future physical therapy session. The one or more actions for the future physical therapy session may be based on several factors including the progression of a patient through a physical therapy plan. The actions are defined for each patient and may also include an assessment of treatment defined outside the virtualized physical therapy system. For example, the actions performed during a physical therapy session along with the healthcare provider feedback on the progression of the patient through a physical therapy plan towards a rehabilitation goal, such as rehabilitation of upper body members, regain walking ability, reduce phantom pain, etc., can be used to determine actions for the future physical therapy session.

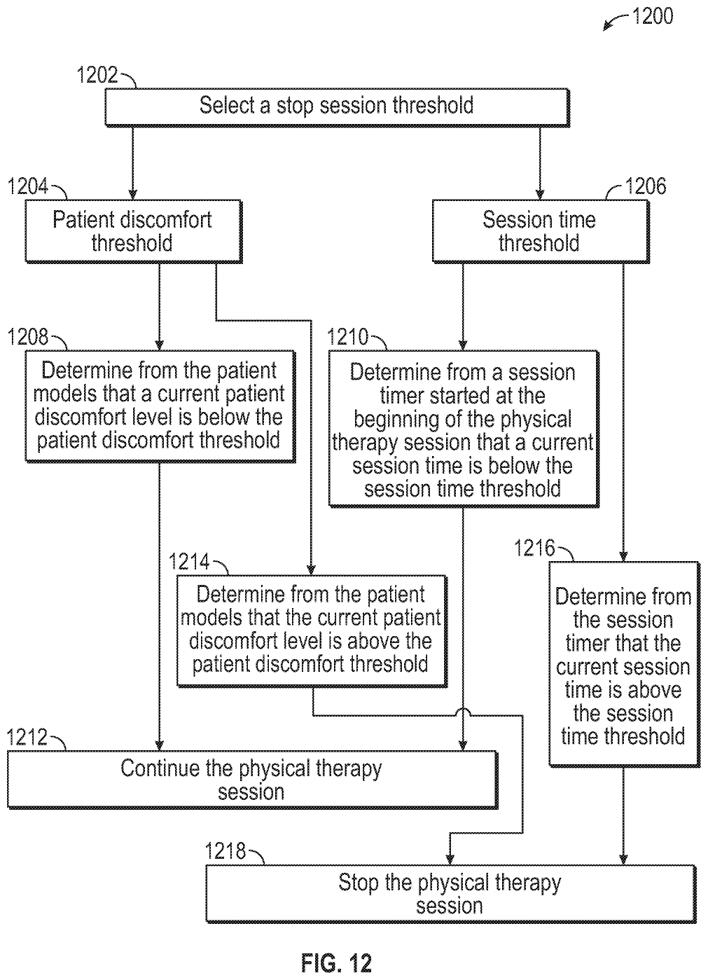

FIG. 12 illustrates an additional method for a virtualized physical therapy system, according to one embodiment. The method 1200 begins at block 1202 where a computer, such as computer 301, during the performance of the selected actions, selects a stop session threshold. In some examples, the method continues when the computer, such as computer 301, continues the physical therapy session upon determining that the stop session threshold has not been met, and stops the physical therapy session upon determining that the continuing session threshold has been met. For example, the method 1200 continues at block 1204 when the stop session threshold selected is a patient discomfort threshold. At block 1208, the computer, such as computer 301, determines from the patient models that a current patient discomfort level is below the patient discomfort threshold and continues the physical therapy session at block 1212. At block 1214, the computer, such as computer 301, determines from the patient models that the current patient discomfort level is above the patient discomfort threshold and stops the physical therapy session at block 1218.

In another example, the method 1200 continues at block 1206 when the stop session threshold selected is a session time threshold. At block 1210, the computer, such as computer 301, determines from a session timer started at the beginning of the physical therapy session that a current session time is below the session time threshold and continues the physical therapy session at block 1212. At block 1216, the computer, such as computer 301, determines from the session timer that the current session time is above the session time threshold and stops the physical therapy session at block 1218.

In the following, reference is made to embodiments presented in this disclosure. However, the scope of the present disclosure is not limited to specific described embodiments. Instead, any combination of the following features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the following aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

Aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.”

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications (e.g., program modules 315) or related data available in the cloud. For example, the virtual PT system module 311 could execute on a computing system in the cloud and interact with the other program modules 315 and data to perform the functions described herein. In such a case, the virtual PT system module 311 could receive, access, update, and store patient data 321, measurement data 322, and action data 323 at a storage location in the cloud. Doing so allows a user to access this information from any computing system attached to a network connected to the cloud (e.g., the Internet).

While the foregoing is directed to embodiments of the present invention, other and further embodiments of the invention may be devised without departing from the basic scope thereof, and the scope thereof is determined by the claims that follow.

What is claimed is:

1. A method comprising:
initializing a virtualized physical therapy system for a patient, the virtualized physical therapy system comprising:
a brain computer interface (BCI) connected to the patient;
a virtual reality component; and
one or more monitoring sensors;
selecting one or more actions to be performed by the patient during a physical therapy session using the virtualized physical therapy system;
determining, using the BCI, that the patient is performing one or more of the selected actions;
displaying, using the virtual reality component, a virtual representation of the selected actions;
monitoring the patient, using the virtualized physical therapy system, during the performance of the selected actions;
updating one or more patient models with monitoring data; and
generating a session evaluation report for the physical therapy session based on the patient models and the monitoring data.

2. The method of claim 1, wherein initializing a virtualized physical therapy system for the patient further comprises:
determining a proper positioning for the brain controlled interface, the virtual reality component, and the one or more monitoring sensors;
indicating the proper positioning to a user; and
performing one or more calibration methods to calibrate the virtualized physical therapy system to the patient.

3. The method of claim 1, wherein selecting the one or more actions to be performed by the patient during a physical therapy session comprises:
selecting the one or more actions from a stored physical therapy plan.

4. The method of claim 1, wherein selecting the one or more actions to be performed by the patient during a physical therapy session comprises:
receiving action input from a user; and
determining from the action input, the one or more actions to perform.

5. The method of claim 1, determining, using the BCI, that the patient is performing one or more of the selected actions further comprises:
receiving an indication from the BCI that the patient is performing a movement; and
determining that the movement corresponds to the selected action.

6. The method of claim 1, wherein the virtual reality component comprises a virtual reality headset and wherein displaying the virtual representation of the selected actions comprises:
determining a virtual representation of the selected action; and
displaying the virtual representation on a display of the virtual reality headset.

7. The method of claim 1, wherein the one or more monitoring sensors comprise biometric sensors configured to read biometric indication from the patient and wherein monitoring the patient, further comprises:
receiving, from the biometric sensors and BCI, data representing a current physical condition of the patient;
receiving additional data from a user indicating the current physical condition and a current psychological condition; and
generating a current monitoring score for the patient using the data and additional data.

8. The method of claim 1, wherein the method further comprises:
during the performance of the selected actions, determining feedback on the performance of the selected actions; and
providing the feedback to the patient using the virtual reality component.

9. The method of claim 1, further comprising:
during the performance of the selected actions, selecting a stop session threshold;
upon determining that the stop session threshold has not been met, continuing the physical therapy session; and
upon determining that the stop session threshold has been met, stopping the physical therapy session.

10. The method of claim 9, wherein the stop session threshold comprises a patient discomfort threshold, and wherein determining that the stop session threshold has not been met comprises:
determining from the patient models that a current patient discomfort level is below the patient discomfort threshold; and
wherein determining that the stop session threshold has been met comprises:
determining from the patient models that the current patient discomfort level is above the patient discomfort threshold.

11. The method of claim 9, wherein the stop session threshold comprises a session time threshold, and wherein determining that the stop session threshold has not been met comprises:
determining from a session timer started at a beginning of the physical therapy session that a current session time is below the session time threshold; and
wherein determining that the stop session threshold has been met comprises:
determining from the session timer that the current session time is above the session time threshold.

12. The method of claim 1, wherein generating the session evaluation report for the physical therapy session based on the patient models and the monitoring data further comprises:
generating a score for the selected actions performed using the one or more patient models; and
determining, from the one or more patient models, one or more actions to be performed for a future physical therapy session.

13. A system, comprising:
one or more computer processors;
a brain computer interface (BCI) connected to a patient;
a virtual reality component;
one or more monitoring sensors; and
a memory containing a program which when executed by the processors performs an operation comprising:
initializing a virtualized physical therapy system for the patient, the virtualized physical therapy system comprising:
the brain computer interface (BCI) connected to the patient;
the virtual reality component; and
the one or more monitoring sensors;
selecting one or more actions to be performed by the patient using the virtualized physical therapy system during a physical therapy session;
detecting, using the BCI, that the patient is performing one or more of the selected actions;
displaying, using the virtual reality component, a virtual representation of the selected actions;
monitoring the patient, using the virtualized physical therapy system, during the performance of the selected actions;
updating one or more patient models with monitoring data; and
generating a session evaluation report for the physical therapy session based on the patient models and the monitoring data.

14. The system of claim 13, wherein the one or more monitoring sensors comprise biometric sensors configured to read biometric indications from the patient and wherein monitoring the patient, further comprises:
receiving, from the biometric sensors and BCI, biometric data representing a current physical condition of the patient;
receiving, from the biometric sensors and BCI, biometric data representing a current physiological condition of the patient; and
generating a current monitoring score for the patient.

15. The system of claim 13, wherein the operation further comprises:
during the performance of the selected actions, determining feedback on the performance of the selected actions; and
providing the feedback to the patient using the virtual reality component.

16. The system of claim 13, wherein the operation further comprises:
during the performance of the selected actions, selecting a stop session threshold;
upon determining that the stop session threshold has not been met, continuing the physical therapy session; and
upon determining that the stop session threshold has been met, stopping the physical therapy session.

17. The system of claim 13, wherein generating the session evaluation report for the physical therapy session based on the patient models and the monitoring data further comprises:
generating a final score for the selected actions performed using the one or more patient models; and
determining, from the one or more patient models, one or more actions to be performed for a future physical therapy session.

18. A computer program product for a virtualized physical therapy system, the computer program product comprising:
a computer-readable storage medium having computer-readable program code embodied therewith, the computer-readable program code executable by one or more computer processors to perform an operation comprising:
initializing the virtualized physical therapy system for a patient, the virtualized physical therapy system comprising:
a brain computer interface (BCI) connected to the patient;
a virtual reality component; and
one or more monitoring sensors;
selecting one or more actions to be performed by the patient using the virtualized physical therapy system during a physical therapy session;
detecting, using the BCI, that the patient is performing one or more of the selected actions;
displaying, using the virtual reality component, a virtual representation of the selected actions;
monitoring the patient, using the virtualized physical therapy system, during the performance of the selected actions;
updating one or more patient models with monitoring data; and
generating a session evaluation report for the physical therapy session based on the patient models and the monitoring data.

19. The computer program product of claim 18, wherein the one or more monitoring sensors comprise biometric sensors configured to read biometric indications from the patient and wherein monitoring the patient, further comprises:
receiving, from the biometric sensors and BCI, biometric data representing a current physical condition of the patient;
receiving, from the biometric sensors and BCI, biometric data representing a current physiological condition of the patient; and
generating a current monitoring score for the patient.

20. The computer program product of claim 18, wherein generating the session evaluation report for the physical therapy session based on the patient models and the monitoring data further comprises:
generating a final score for the selected actions performed using the one or more patient models; and
determining, from the one or more patient models, one or more actions to be performed for a future physical therapy session.
